(19) United States
(12) Patent Application Publication
(10) Pub. No.: US 2003/0142269 A1
(43) Pub. Date: Jul. 31, 2003

(54) Device for immersion biometry

(76) Inventor: J. Stuart Cumming, Laguna Beach, CA (US)

Correspondence Address: FULBRIGHT & JAWORSKI, LLP, 1301 MCKINNEY, SUITE 5100, HOUSTON, TX, 77010-3095, US

(21) Appl. No.: 10/058,481

(22) Filed: Jan. 28, 2002

(51) Int. Cl. A61B 3/00

(52) U.S. Cl. 351/200

(57) ABSTRACT

A biometry device to facilitate immersion biometry allowing the biometrist to perform the study without having to hold a device onto the patient's eye and without the need for a local topical anesthetic. The device consists of a mask or goggles that fit over the patient's eyes. The mask/goggles can have a single fluid chamber covering both eyes or two fluid chambers, one to cover each eye. Transducers are mounted in the goggle, one opposite each eye. The transducers are mounted in a housing that allows each one to be manipulated so that it is axially aligned with the eye under examination.

### BACKGROUND OF THE INVENTION

Intraocular lenses were first implanted following cataract surgery by Harold Ridley in England in 1949. Since then there have been enormous advances in both the lens design and the surgical procedure for removal of cataracts. The modem surgical procedure is done under topical anesthesia, the cataract is removed by phacoemulsification and a foldable replacement lens is inserted into the eye through a 3.5 mm incision. The surgical procedure takes approximately 10-15 minutes and the patient goes home in less than an hour.

An appropriate intraocular lens of the correct power has to be selected for each individual eye undergoing surgery. In the early days of lens implantation this was based upon the patient's refraction. Later ultrasound biometry was developed to further refine the true accuracy of lens power selection. In the majority of cases this is performed by placing a transducer on the surface of the cornea and recording by means of a printer the peaks of the sound wave as it strikes the posterior inner surface of the eye, the posterior and anterior surfaces of the human lens and the anterior and posterior surfaces of the cornea. From these tracings the axial length of the eye, the length of the vitreous cavity, the lens thickness and the depth of the anterior chamber can be measured. One or a combination of these measurements are fed into a computer along with measurements of the average of the radii of curvature of the cornea at both its steepest and flattest meridians, the “K” Readings.” The biometrist then enters into the computer a constant for the particular lens design (the “A” constant) and selects one of several available formulas to calculate the lens power. The computer printer then produces a list of lenses for selection with the anticipated postoperative refraction for each lens power. The surgeon then selects from the computer printout the appropriate lens to implant.

This technique of biometry is by far the most common currently performed method of biometry and requires a skilled and experienced technician. The technician has to place the transducer onto the cornea with the least amount of corneal distortion or flattening and do this consistently from eye to eye. The most critical measurement entered into the formula is the axial length of the eye. A 1 mm indentation of the cornea shortens the axial length, and can result in a postoperative refractive error of 3 diopters leaving the patient severely myopic. Accurate biometry is therefore vital to get good uncorrected visions postoperatively. Many surgeons have not, until recently, evaluated the uncorrected outcomes of their surgery.

In the late 90's a multifocal intraocular lens was introduced into the market, the Array™ by Allergan. This lens focuses the light on the retina of the eye both for distance and near simultaneously. The brain has to select the appropriate image it wishes to recognize. This lens allows the patient to see at distance and near; however because the light in focus is divided between a distance and near target, contrast is lost and there is significant glare. The development of this lens, however, has made the eye surgeon conscious of the importance of the accuracy of the preoperative biometry. Since the objective of implanting the lens is to enable the patient to live without glasses. As explained above, this examination entails utilizing sound waves to measure the length of the eye, this measurement plus the radii of curvature of the cornea at its steepest and flattest meridians is applied to one of several formulas to determine what lens power should be implanted into the eye to give a predetermined preoperative refraction. In most cases this selection of the lens power for an eye would result in the patient being able to see well as distance without glasses, i.e. emmetropia.

The introduction of the Array™ intraocular lens should enable patients to see at distance and near without glasses. In order to achieve this goal the surgeon has to have excellent uncorrected vision and the importance of accurate biometry has become very apparent to the surgeons implanting this multifocal lens.

Accurate biometry can be achieved by two methods, one utilizing the standard biometry equipment modified to allow the measurement to be made through a fluid or water bath (immersion biometry). The second method is by means of the IOL Master™ from Zeiss which utilizes partial coherent laser interferometry to define the various intraocular measurements. Immersion biometry has not been popular with surgeons and their technicians because a chamber has to be placed onto the eye and filled with fluid before the biometry measurement can be made. The techniques for doing this have been cumbersome and in many cases required the patients to lie flat on their backs and the technician to be skilled. The great advantage of this technique however is that there is not corneal distortion and therefore very accurate lens power estimations care obtained. The IOL Master™ from Zeiss is also accurate because it is user friendly and does not involve contact with the cornea. This instrument does, however, have two disadvantages. First, it cannot be used on patients with dense cataracts and secondly is expensive. The immersion technique, if it could be simplified, is therefore the preferable technique. The basis of this patent is a simplification of the technique of immersion biometry.

### BRIEF SUMMARY OF THE INVENTION

The invention provides a device to facilitate immersion biometry allowing the biometrist to perform the study without having to hold a device onto the patient's eye and without the need for a local topical anesthetic. The device renders the examination much more acceptable and more comfortable to the patient than other immersion biometry techniques.

The device consists of a mask or goggles that fit over the patient's eyes. The mask/goggles can have a single fluid chamber covering both eyes or two fluid chambers, one to cover each eye. The mask, designed to be watertight, is held in place by an adjustable strap that goes around the head. The mask is connected by flexible tubes to a container or bag of fluid, usually water or saline.

The container is connected by one tube if there is one chamber in the goggle or two tubes if there are two chambers. The fluid container is located beneath the goggle. The chamber(s) of the goggle is (are) filled with saline by raising the bag above the level of the goggle. When the goggle chambers are full of saline, a clamp is closed across the tube connecting the container to the goggle and the bag lowered to a level beneath the goggle. There is then a “waterbath” between the eye and the front of the goggle through which sound waves can pass without there being any contact between the goggle fitted with a transducer and the eye.

Two transducers are mounted in the goggle, one opposite each eye. The transducers are mounted in a housing that allows each one to be manipulated so that it is axially aligned with the eye under examination. Each eye is examined separately by re-routing the cables connecting each transducer to the ultrasound scanner.

Upon completion of the scans, the clamp preventing the drainage of saline from the goggle is opened and the saline is drained back into the bag.

The parallel-sided transducer is mounted in the goggle such that its alignment can be moved to align with the optical axis of the eye. This is essential in order to take a reading. The adjustable alignment can be achieved by a ball-and-socket arrangement or XY movements. The transducer has to be movable in and out to a distance range in which measurements can be taken. The housing or opening for the transducer has to be watertight, which can be achieved by the use of a circular flexible membrane with a hole in its center or with O rings. The transducer can then slide into or out of the mask or goggles and rotate onto the optical axis by asking the patient to look at a fixation light on the end of the transducer. During the manipulation, the examiner would watch the biometer screen; a measurement would only be recorded when the transducer distance and alignment with the eye is correct. The cables from each transducer would be activated alternately by a cable switch such that one eye could be examined after the other.

The mask or goggle design has a hole(s) in its roof to allow the air to escape from the mask as it fills with saline or water. The floor of the mask tapers or funnels into the outflow tube so that when the clamp or lock is opened the saline will pool in the funnel during its drainage from the mask, leaving little or no residue.

The mask is constructed such that it has windows through which the biometrist can observe the tip of the transducer to correctly position it relative to the eye. Alternatively the mask may utilize a goggle, similar to a swimming goggle, with a transducer mount

The mask will fit all faces and have an adjustable headband to fit around or around and over the head.

A system can be designed such that there are two bags: the input bag and the drain bag. The goggles are filled by lifting the input bag above the level of the goggles after opening a clamp or a stopcock. After the goggles are full, the clamp is closed and the bag with a reservoir of remaining solution is lowered to rest on the patient's lap. Solution remaining in the bag can be used for future examinations. After completing the biometry, a second clamp or stopcock is opened and the fluid drained from the goggle.

Utilizing the system with cleaning the goggles between patients, multiple examinations can be performed using the same input bag of fluid.

### BRIEF DESCRIPTION OF THE DRAWINGS

For a more complete understanding of the present invention, reference is now made to the following descriptions taken in conjunction with the accompanying drawing, in which:

FIG. 1 is a perspective frontal view of one embodiment of the biometry goggles;

FIG. 2 is a perspective top view of the biometry goggles of FIG. 1;

FIG. 3a is a perspective frontal view of one embodiment of the biometry with viewing windows;

FIG. 3b is a perspective frontal view of an alternative embodiment of the biometry goggles with viewing windows;

FIG. 4 is sectional side view of one embodiment of a fluid chamber with a ball-and-socket system;

FIG. 5 is a sectional side view of the fluid chamber of FIG. 4 further illustrating the ball-and-socket system;

FIG. 6 is a top view of the fluid chamber of FIG. 5;

FIG. 7 is schematic top view illustrating an alternative embodiment of the invention with an X-Y system;

FIG. 8 is a schematic illustrating an embodiment of the invention utilizing a single fluid container; and

FIG. 9 is a schematic illustrating an embodiment of the invention utilizing a first fluid container and a second fluid container.

### DETAILED DESCRIPTION OF THE INVENTION

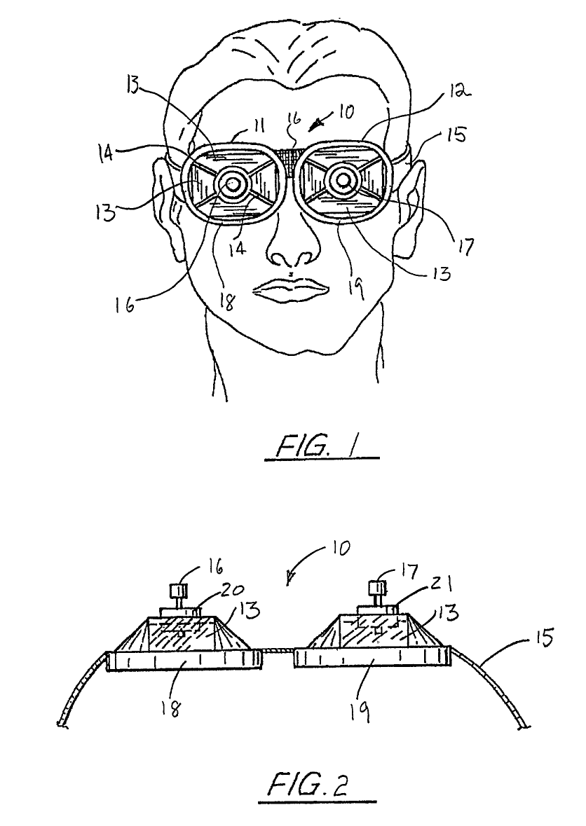

Referring to FIG. 1, a pair of biometry goggles 10 is shown worn about a patient's head. In the displayed embodiment, a pair of fluid chambers or goggles 11, 12 are shown placed over the patient's eyes. The fluid chambers 11, 12 are interconnected to one another through a fluid chamber connector 16. This fluid chamber connector may be a flexible strap, rigid piece of material connecting the fluid chambers 11, 12, such as plastic, or the fluid chamber connector may be made of any material allowing for adjustable positioning of the fluid chambers 11, 12 over the patient's eyes. Furthermore, the fluid chamber connector may be integrally molded with the fluid chambers 11, 12. The fluid chambers 11, 12 are secured to the patient's head preferably with a strap 15 that is flexible and adjustable. In other embodiments, other materials and other configurations may utilized to secure the fluid chambers 11, 12 over the patient's eyes. Although the biometry goggles 10 are shown in FIG. 1 with two fluid chambers 11, 12, an alternate embodiment of the invention includes a single fluid chamber. This single fluid chamber may be a chamber that covers one or both eyes of a patient.

The fluid chambers 11, 12 have a fluid chamber base 18, 19. The fluid chamber base 18, 19 interfaces with the surface of the patient's head. The fluid chamber base 11, 19 may be configured concavely to follow the curvature of a person's head. The fluid chamber base 18, 19 provides a water tight seal around the patient's eye. The fluid chamber base may include a rubber seal, a sponge foam, or other material to form a seal between the fluid chamber base and the patient's head, for example material that is ordinarily used in swimming goggles to form a seal around the eyes.

In one embodiment the fluid chambers 11, 12 of the biometry goggles 10 have a viewing panel/transducer support frame 14 and one or more viewing panels 13. The viewing panel 13 are fitted within the support frame 14 and the fluid chamber base 18, 19. The viewing panels allow the device operator to see the patient's eye and to view the positioning of the transducer 16. Alternatively, the fluid chamber may be made with a single viewing panel or goggle with the viewing panel or goggle having a central transducer mount. The viewing panels or goggle are preferably made from a polycarbonate material. However, other material may be used that allowing viewing through the material, for example, certain plastics and glass.

Referring now to FIG. 2, a perspective top view of the biometry goggles of FIG. 1 is shown. The fluid chambers 11, 12 have transducers 16, 17 that are mounted in the fluid chambers. The fluid chambers 11, 12 have a transducer mount 20, 21. The transducer mount 20, 21 houses the transducer 16, 17. The transducer mount 20, 21 allows for movement of the transducer 16, 17 for biometry analysis of the eye.

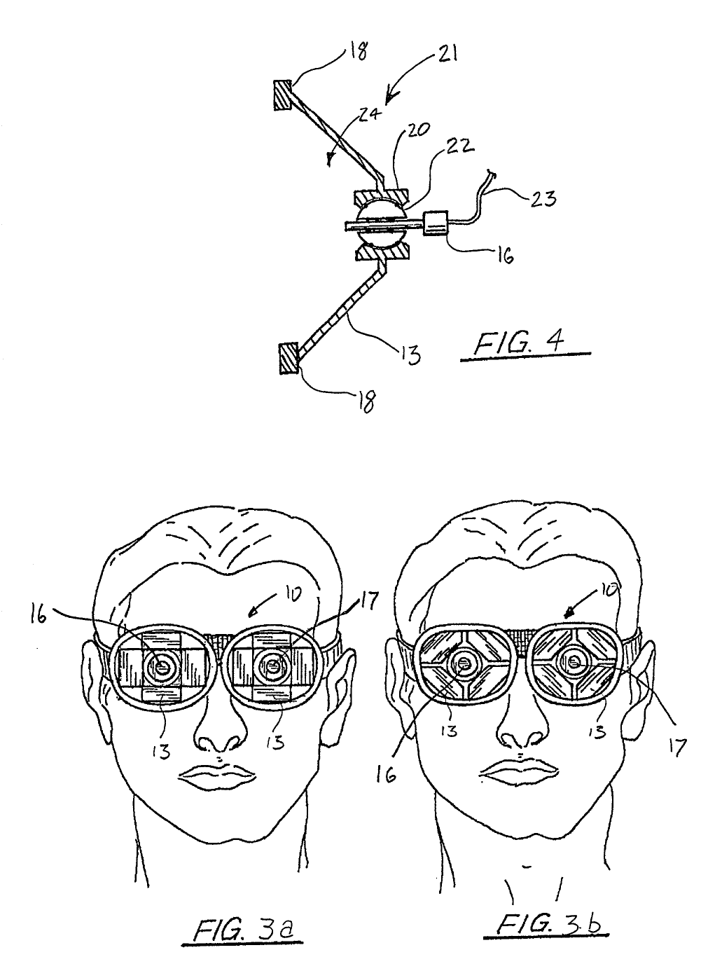

FIGS. 3a and 3b illustrate other embodiments of the biometry goggles 10 showing different configurations of viewing panels.

FIG. 4 is a sectional side view of one embodiment of a fluid chamber 21. In this embodiment, the transducer mount 20 utilizes a ball 22 for rotatable movement of the transducer 16. A transducer lead 23 is connected to the transducer 16. The transducer 16 is held within the rotatable ball 22. The fluid chamber 21 when placed over the patient's eye forms a “liquid-tight” fluid reservoir 24. The transducer 16 when taking biometry readings is immersed in the fluid reservoir.

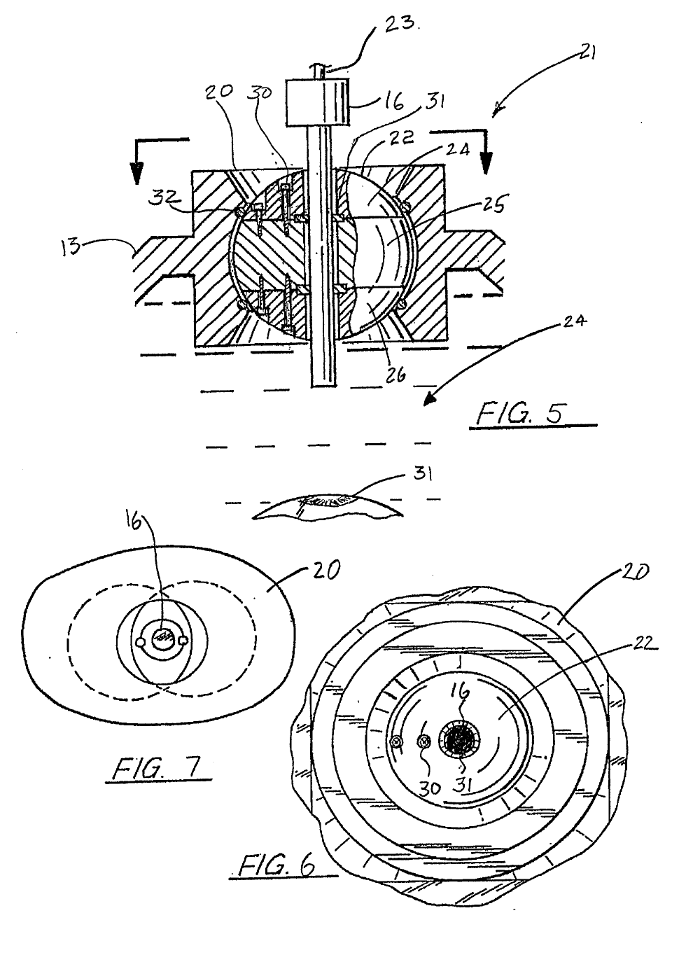

Referring now to FIG. 5, a sectional side view of the fluid chamber of FIG. 4 further illustrates the ball-and-socket system. The ball 22 of the ball and socket system holds the transducer 16 in place. The transducer 16, in addition to being rotatably positionable, may be moved anteriorly away from the surface of the eye 31 and posteriorly towards the surface of the eye 31. The ball 22 may be configured in separate sections such that the ball is removable from the transducer mount 20. In this configuration, the ball includes an anterior portion 24 and posterior portion 25 which are held to a main body 26 of the ball 22 by set screws or other fixation means. Seals 32, 31 may be used to provide a water-tight seal for the transducer. The seals 31 may be replaced by removing the anterior or posterior portions 24, 26 and exchanging the seal for a new seal.

FIG. 6 is a top view of the fluid chamber of FIG. 5. The transducer 16 is placed in a central hole of the ball 22. In this embodiment, a rubber seal 16 is utilized to retain the transducer 16 in the ball 22 and allow the transducer to move anteriorly and posteriorly from the eye while providing a water-tight seal.

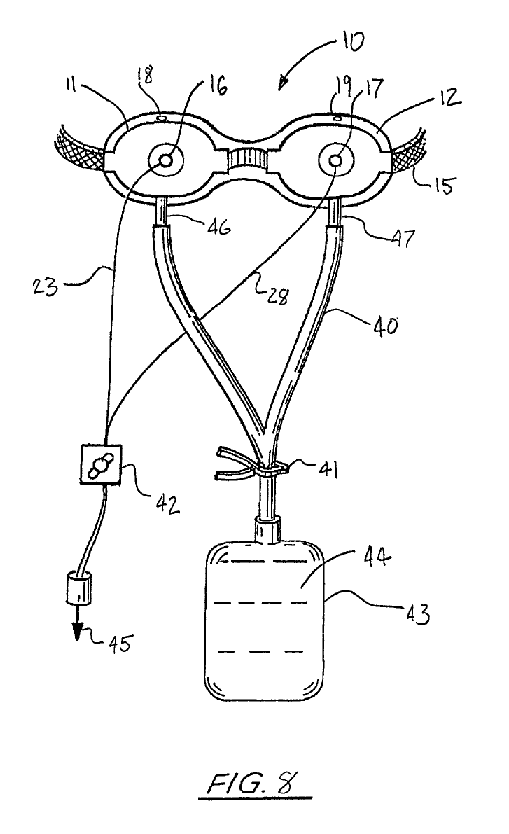

In FIG. 7 an alternate embodiment of the transducer mount 20 is shown. In this embodiment the transducer mount 20, includes an X-Y visual axis alignment mechanism instead of the rotating ball system. The X-Y mechanism allows the operator to examine the eye in the straight-ahead position adjusting the transducer up or down and side to sided to correspond to the visual axis of the eye.

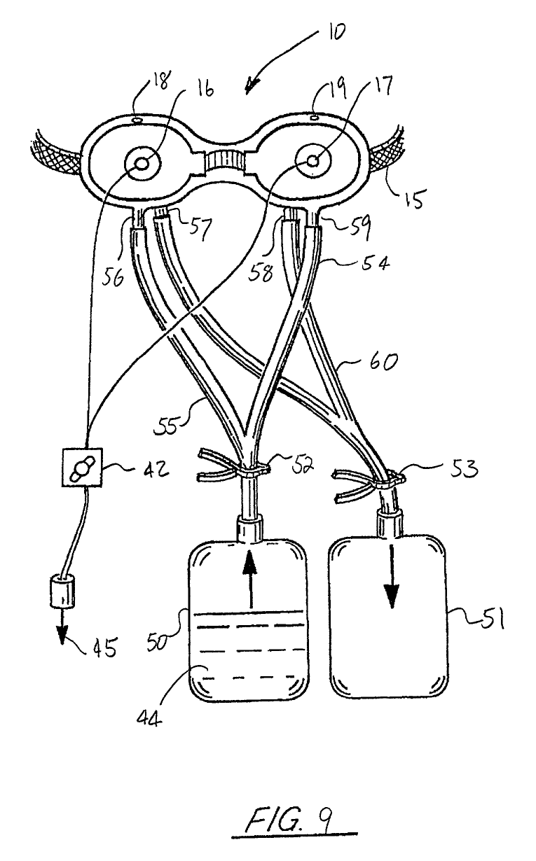

Referring now to FIG. 8, a schematic illustration depicts an embodiment of the biometry goggles 10 utilizing a single fluid reservoir 43 for providing a fluid 44 to the fluid chambers 11, 12. A patient wearing the biometry goggles generally lies in the supine position. The fluid reservoir 43 is raised to a level above the biometry goggles 10. A fluid flow regulator 41, such as a clamp or stopcock, controls the release of the fluid 44 from the fluid container 43. A fluid transport carrier 40, such as tubing, attaches to the biometry goggles to fluid valves 46 and 47. The fluid flow regulator 41 when opened releases the fluid 44 from the fluid reservoir 43 which is then dispensed into the fluid chambers 11, 12. When fluid chambers 11, 12 are full of fluid, the fluid flow regulator 41 is closed and the fluid reservoir 43 lowered to a level beneath the goggles 10. The fluid in the fluid chambers 11, 12 provides a “waterbath” between the eye and the transducers 16, 17. Sound waves can pass through the fluid without any contact between the eye and the transducers 16, 17. After biometric readings have been taken, the fluid in the fluid chambers 11, 12 may be released back to the fluid reservoir 43.

The biometry goggles 10 may include air inlet/outlet valves 18, 19 to release air in the fluid chambers 11, 12 when filing the chambers with the fluid 44 and to intake air when releasing the fluid back into the fluid reservoir 43.

Transducer lead lines 23, 27 may be connected to a control switch 42 allowing for selective biometric reading for an individual transducer, or both, allowing the operator to take independent readings of the right eye or the left eye, or both concurrently. The lead lines 23, 27 continue through the control switch 42 to a plug 45 for the biometric reading machine.

Referring to FIG. 9, a schematic illustration of an embodiment of the invention is shown where the biometry goggles 10 utilize a first fluid reservoir 44 and a second fluid reservoir 51. In this embodiment, a first fluid reservoir 55 containing a fluid 44 is release by way of a first flow control regulator 52 into to the biometry goggles 10. This operation is similar to the operation of the embodiment of FIG. 8. Instead of the fluid being released back into the same reservoir, when biometric readings are completed, the fluid is released into a second fluid reservoir 51. The release into the second fluid reservoir is controlled by a second flow control regulator 53. In this embodiment, each fluid chamber includes two fluid valves 56, 57, and 58, 59. A first set of fluid valves 56, 58 are used to fill the fluid chambers 11, 12 from a first fluid transport carrier 55, and a second set of fluid valves 57, 58 are used to release the fluid into a second fluid transport carrier 60.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A biometry eye device comprising:
at least one fluid chamber for covering one or both eye's of a person, said at least one fluid chamber having a transducer mount; and
a transducer attached to each transducer mount for taking biometric reading of a person's eye.

2. The biometry eye device of claim 1, wherein the at least one fluid chamber has a fluid opening for filling or draining fluid.

3. The biometry eye device of claim 2 further comprising:
a fluid reservoir having a fluid flow regulator and fluid transport carrier, wherein the fluid transport carrier attaches to the fluid opening.

4. The biometry eye device of claim 1, wherein the at least one fluid chamber includes a fluid inlet valve and a fluid outlet valve.

5. The biometry eye device of claim 4 further comprising:
a first fluid reservoir for providing a fluid to the fluid inlet valve; and
a second fluid reservoir for receiving the fluid from the fluid outlet valve.

6. The biometry eye device of claim 1, wherein the at least one fluid chamber when worn by a person forms a seal about the person's face to prevent fluid leakage.

7. The biometry eye device of claim 1, wherein the at least one fluid chamber has one or more transparent windows.

8. The biometry eye device of claim 1, wherein the transducer mount is positioned in the fluid chamber such that the transducer is located over the eye of the person when the device is worn by the person.

9. The biometry eye device of claim 1, wherein the transducer includes a fixation light.

10. The biometry eye device of claim 1, wherein the transducer is detachably mounted to the fluid chamber.

11. The biometry eye device of claim 1, wherein at least one fluid chamber is configured in the form of a single goggle or double goggles.

12. The biometry eye device of claim 9, further comprising:
an adjustable strap connected to the at least one fluid chamber.

13. The biometry eye device of claim 1, further comprising:
a visual axis alignment mechanism for aligning the transducer to the visual axis of the eye.

14. The biometry eye device of claim 13, wherein the visual axis alignment mechanism is an X-Y system.

15. The biometry eye device of claim 13, wherein the visual axis alignment mechanism is a rotational ball system.

16. The biometry eye device of claim 14 or claim 15, wherein the transducer is adjustable in and out of the at least one fluid chamber for positioning the transducer to selected distance from the cornea of the eye.

17. The biometry eye device of claim 13 wherein the alignment mechanism is adapted to lock the transducer in place.

18. The biometry eye device of claim 1, wherein the at least one fluid chamber is made from material that can be sterilized.

19. The biometry eye device of claim 1, wherein at least one fluid chambers are supported by a support frame configured in the form of a single goggle or double goggles.

20. A biometry eye device comprising:
a first and second fluid chamber configured to be worn on a person's head to cover the person's eyes, and
a first transducer attached to the first fluid chamber and a second transducer attached to the second fluid chamber, wherein the first and second transducer may be independently aligned with the visual axis of each eye.

21. The biometry eye device of claim 20 further comprising:
a switch operable with the first and second transducer such that the left eye and right eye can be examined sonometrically or be examined consecutively.

22. The biometry eye device of claim 20, further comprising:
a visual axis alignment mechanism for aligning the first and second transducer to the visual axis of the left eye and right eye.

23. The biometry eye device of claim 20, further comprising:
a visual axis alignment mechanism for aligning the transducer to the visual axis of the eye.

24. The biometry eye device of claim 23, wherein the visual axis alignment mechanism is an X-Y system.

25. The biometry eye device of claim 23, wherein the visual axis alignment mechanism is a rotational ball system.

26. The biometry eye device of claim 24 or claim 25, wherein the transducer is adjustable in and out of the at least one fluid chamber for positioning the transducer to selected distance from the cornea of the eye.

27. A biometry eye device comprising:
a support frame configured in the form of goggles to be worn on a person's head;
at least one fluid chamber supported by the support frame such that the at least one fluid chamber is positioned over a person's eye when the support frame is worn by a person, the at least one fluid chamber having a fluid opening for filling fluid into the fluid chamber and/or draining fluid from the fluid chamber,
a transducer attached to the at least one fluid chamber; and
a visual axis alignment mechanism for aligning the transducer to the visual axis of the eye.

28. The biometry eye device of claim 27 further comprising:
at least one fluid reservoir adapted for attachment to the fluid opening.

29. The biometry eye device of claim 27, wherein the at least one fluid chamber has one or more transparent windows.

30. The biometry eye device of claim 27 further comprising:
a switch operable with the transducer for operation of the transducer.

31. The biometry eye device of claim 27 further comprising:
an adjustable strap connected to the support frame.

32. The biometry eye device of claim 27, wherein the transducer includes a fixation light.

33. A method of immersion biometry comprising the steps of:
applying a biometry eye device to the head of a person, the biometry eye device comprising at least one fluid chamber, wherein the fluid chamber is positioned over the person's eye when the device worn on the person's head, and a transducer attached to the at least one fluid chamber; and
obtaining a biometry reading of the person's eye or eyes utilizing the biometry eye device.

34. The method of claim 33, further comprising the step of filling the at least one fluid chamber of the biometry eye device with a fluid.

35. The method of claim 34, further comprising the step of draining the fluid from the at least one fluid chamber into a water bath or into a fluid reservoir.

36. The method of claim 33, further comprising the step of aligning the transducer with the visual axis of the person's eye.

37. The biometry eye device of claim 33, wherein the at least one fluid chamber has one or more one transparent windows.

38. The biometry eye device of claim 33, wherein the transducer is detachably mounted to the fluid chamber.

39. The biometry eye device of claim 33, wherein fluid chambers are supported by a support frame configured in the form of a single goggle or double goggles.

40. The biometry eye device of claim 39, wherein the support frame is adjustable to accommodate differing head sizes.
